(19) United States
(12) Patent Application Publication
(10) Pub. No.: US 2014/0311227 A1
(43) Pub. Date: Oct. 23, 2014

(54) PORTABLE DEVICE FOR MEASURING BLOOD SAMPLES

(71) Applicants: Salla Koski, Helsinki (FI); Mikko Tasanen, Helsinki (FI)

(72) Inventors: Salla Koski, Helsinki (FI); Mikko Tasanen, Helsinki (FI)

(73) Assignee: MODZ OY, Helsinki (FI)

(21) Appl. No.: 14/362,177

(22) PCT Filed: Dec. 1, 2011
(86) PCT No.: PCT/FI2011/051069

§ 371 Date: Jun. 2, 2014

(51) Int. Cl. G01N 33/66

(52) U.S. Cl. CPC G01N 33/66
USPC 73/61.41

(57) ABSTRACT

The invention relates to a device for measuring and monitoring glucose content of blood. The invention is especially applicable for users with diabetes disease. In a device of the present invention sample input aperture (24) can be blocked when it is not in use. The blocking can be implemented with a sliding cover (21), which blocks either the input for the sample strip (24) or the connection for a data cable (11). It is possible to prevent impurities accessing the sensor of the measurement unit (14), and obtain required electrical isolation when a data cable is connected to the electrical connection (11). In addition to measurement functions, the device may have functionalities for monitoring diet or even for playing games.

### FIELD OF INVENTION

The invention relates to a portable device for measuring blood samples, such as glucose content of blood samples. The invention is especially applicable for users who have diabetes disease.

### BACKGROUND TECHNOLOGY

A person with a diabetes disease must take especially good care for his/her diet, general condition, regular rest and medication. The care of the diabetes disease also demands regular concentration measurement of blood sugar i.e. blood glucose. The measurement must be performed even several times a day. The measurement commonly takes place by taking a small sample of blood from a finger tip, which sample is let to absorb into a sample test dot of a sample test strip. The glucose concentration is measured from the strip with a portable measurement device, for example. Such a device according to prior art is described e.g. in publication WO2008/62099A1. A measurement device is used by placing a sample test strip to the measurement device and setting a blood sample on the test strip, after which the measurement device performs blood glucose measurement from the blood sample, and shows the measurement result on the display of the measurement device.

When a person knows the glucose concentration the person may assess, whether he/she has need for an instant care action. If the measured blood glucose concentration is too high, e.g. more than 15 mmol/l, the care action is generally adding insulin to blood by punching. If the measured blood glucose concentration is too low, e.g. less than 3 mmol/l, the care action is generally taking drink or food which contains carbon hydrate. In addition to assessing an instant care action the blood glucose concentration gives information on requirement of eating, physical exercise, rest or medication. The measured data may be input to a computer for an analysis performed by a program.

There also exist systems, by which it is possible to transfer measurement results of the measurement devices to user's work station and possibly further to personnel of the health care, and to perform analyses on the information. Such systems give information on the history of the measurement results.

There are certain problems related to the prior art portable measurement devices. The blood sample is measured by detecting an electric charge, which is created by a chemical reaction. The measurement unit the reads the electric current profile created by the charge. It is therefore important that the electrical contacts of the measurement unit remain clean from impurities. The measurement unit may also read optically calibration data from the sample strip, whereby the optics must also remain clean. Further, the sample strips are sensitive to moisture. Therefore, a measurement device must normally be kept in a specific bag or box. This means that it is often difficult carry the measurement device along. As a consequence, it is possible that the user does not always have the device when it is needed. On the other hand, if the device is kept in a pocket, for example, impurities cumulated in the measurement unit may cause the device to give inaccurate readings.

The prior art devices may have a connection for transferring data with a cable to/from a computer or other external device. However, a computer is not a medical device, and the electrical isolation is not sufficient for devices of medical use. This often means that it is not possible to use a standard cable between the device and a computer, and additional isolation circuits are required. Without such arrangements, there might not be sufficient isolation between mains voltage of a computer and a user who applies a test strip to the measurement device.

Further, the prior art devices provide measurement information but they are not helpful in planning and following user's diet, for example.

Since diabetes is a common disease, insufficient care is a significant problem for the person himself/herself but also for the society. Therefore, the improvement of care and solving the above disadvantages of the prior art devices is important.

### SUMMARY OF THE INVENTION

The object of the invention is to provide a new device for measuring blood samples such as blood glucose levels, to provide information which assists a user in daily care, and by which solution the above problems can be decreased or avoided.

The object of the invention can be achieved with a user portable measurement device, which comprises a measurement unit for measuring blood samples, wherein the measurement unit has an input for providing the blood sample in the measurement unit, and a user interface for presenting to a user information which is based on a measurement result, the user interface comprising at least a display and/or means for forming sound and/or vibration, which device is characterised in that the measurement device means for blocking the sample input during time when the sample input is not under use.

Some preferable embodiments of the invention are described in the dependent claims.

A measurement device according to invention preferably has means for data transfer between the device and a computer or other external device. Means for wired data transfer may include an electrical connector, such as a micro-USB connector, for a data transfer cable between the device and the computer.

According to a preferable embodiment of the invention, the blocking means covers the sample input aperture of the device when the blocking means is in the position for blocking the sample input. The blocking means can be arranged to block the electrical connector when the sample input is not blocked. In a further embodiment the blocking means is a sliding cover, which in its first position covers the sample input, and in the second position covers the electrical connector.

According to one embodiment of the invention the device comprises stored information relating to measurement time table, and the device gives a reminder for the user at a moment when a measurement is to be performed. It is also possible that the device gives instructions for the user on the basis of the measurement results.

According to a further embodiment the measurement device has means for lighting the sample input. The input light may be switched ON when the device gives a reminder for measurement.

In one embodiment of the invention the device has a processor which is adapted to control measurement and reminder functions, and optionally data transfer functions. The device may also have stored information on carbon hydrate contents and/or calorie contents of various foodstuff, and processor means for calculating carbon hydrate contents and/or calorie contents of user defined food. In one further embodiment of the invention the processor means for controlling measurement and processor means for calculating food contents are substantially same processing means.

According to one embodiment of the invention the measurement device has a communication unit for wireless communication with a wireless communication network such as a mobile cellular communication network. The measurement device may be adapted to transfer measurement data, user input data and/or reminder data to a wireless terminal via the wireless network. In one further embodiment the communication unit has an antenna for wireless communication and the antenna and the measurement unit are positioned at opposite ends of the device housing in order to avoid mutual interference.

In one further embodiment the measurement device has means for receiving and storing information from other measurement devices, such as a device which measures the movement of the user, or a pulse meter measuring data of heart pulse rate. The reception means may preferably be radio receivers, such as a NFC or Bluetooth receiver.

It is further possible that the device according to the invention includes stored games which can be played with the device.

The present invention and its embodiments offer substantial advantages over prior art solutions. When the sample input can be blocked with a cover, for example, impurities do not have access to the measurement unit and its sensors. It is thus possible to keep the measurement device in a pocket, for example, and it is easy to always carry the device along. This makes it possible for the user to perform required measurements and possible other advised care whenever needed. Optimal care is thus achieved.

It is possible that the sample input is always blocked when the data transfer cable of a computer is connected to the measurement device. It is therefore not possible to insert a sample strip to the sample input when the data cable is connected. Therefore, no additional electrical isolation is needed, and standard cables, such as USB cables, can be used for data connection between a computer and the measurement device. The measurement device is thus electrically safe in all configurations of use.

It is possible to use a device according to an embodiment of the invention for checking the contents of carbon hydrate and calories of various foodstuff. It is also possible to store in the device information on the content of user's meals. This way the device also assists the user in planning meals and monitoring the user's diet. It is also advantageous that the same portable device can be used for both measurements and planning/monitoring meals. Especially, when same processor and possibly same memory is used for providing both functions, the device can be produced with a small amount of complex components and thus with a low cost.

In this patent application “measurement device” means a device for measuring blood samples, such as glucose concentration and/or other quantities. A measurement device may also provide monitoring functions, reminder functions, data processing functions, data transfer functions, database functions, game functions etc.

“Measurement unit” means in this patent application a part of a measurement device, which has required measurement sensor(s) for measuring blood samples, such as glucose content of a blood sample. The measurement unit may preferably have means for defining a value of a measurement result on the basis of a signal received from the measurement sensor(s). The measurement unit may thus comprise a processor, memory and programs for providing control and calculation functions of the measurement unit.

“A sample substrate” means in this patent application e.g. a strip, which has sample test dots for absorption and measurement of a blood sample.

“A user of a measurement device” means in this patent application primarily a person, whose blood samples are measured with the measurement device.

### BRIEF DESCRIPTION OF DRAWINGS

In the following the invention is described with reference to the enclosed drawings, in which:

FIG. 1 illustrates a block diagram of an exemplary measurement device according to the invention;

FIG. 2a illustrates a bottom view of an exemplary measurement device according to the invention;

FIG. 2b illustrates a perspective view of an exemplary measurement device according to the invention;

FIG. 3 illustrates a back view of the internal assembly of an exemplary measurement device according to the invention; and

FIG. 4 illustrates some other system components which may be in communication with the measurement device according to the invention.

### DETAILED DESCRIPTION OF PREFERRED EMBODIMENTS

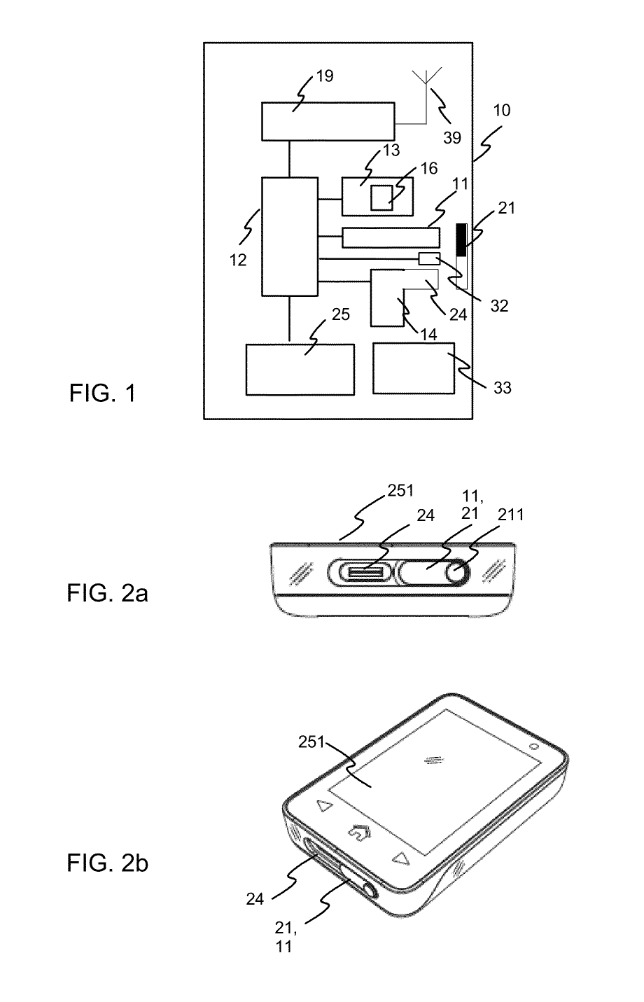

FIG. 1 illustrates a block diagram of an exemplary measurement device according to the invention. The measurement device 10 includes a measurement unit 14 for measuring a blood sample. In the measurement device illustrated in FIG. 1 the measurement unit 14 measures blood glucose concentration from a blood sample which is absorbed into a sample test dot. The measurement unit has an input aperture 24 for placing a test strip, on which a blood sample is then absorbed. The measurement unit is preferably based on electrical charge measurement in a manner, which is prior known as such. The measurement unit preferably includes an amplifier, an analogue-digital converter and other required electronics so that the signal received from the sensor can be fed to the input of a main processor 12 of the device. The processor preferably saves the measurement results into a memory 13 for later use. Also programs 16 controlling the processor have been stored into the memory 13 of the measurement device. Further, data relating to the care program of the user is stored, which data is preferably user specific.

The device of FIG. 1 also has a light emitter 32, such as an LED for providing light in the sample input. The light emitter is preferably controlled with the processor 12. The light may be switched ON when measurement procedure of a blood sample is started by the user. It is also possible that the light is switched ON when the device reminds the user of performing a measurement. The light may be switched OFF after the measurement has been performed.

The measurement device also has user interface means 25, which preferably comprise a display, such as a touch screen. Measurement results can be displayed on the touch screen in text/numbers and/or illustrated with avatars or symbols, for example. The user interface of the measurement device preferably also has means for producing voice and/or vibration. They may produce tones or speech, by means of which the user is guided and given information. The audio signals corresponding to tones or speech can be preferably formed in the processor by means of data stored in the memory. The user interface may also include other input means, such as press button switches, in addition to the touch screen input.

The measurement device has data transfer port 11, by means of which it is possible to transfer data between the measurement unit and a computer or other equipment of a user. Through the data connection it is possible to transfer measurement results and other user data which are stored by the measurement device to a terminal equipment and/or to transfer programs or user data from the computer to the measurement device. The data transfer takes place in a wired manner, e.g. via a micro-USB (Universal Serial Bus) port/connector. Such an electrical connector may be used for other purposes as well, such as connecting to other measurement devices, or charging the battery of the device with a charger or from a USB connection of a computer, for example. It may also be possible that the connection 11 of the measurement device can connect to a data transfer network, whereby it is possible to transfer data with another device which is in connection via the network.

The measurement device also has blocking means 21, such as a sliding cover. The cover has two positions, where in a first position it covers the sample input, and in the second position it covers the electrical connector. One purpose of the cover 21 is to prevent using the electrical connector and the sample input at the same time. This way a proper electrical isolation is provided. Another purpose of the cover is to prevent impurities from accessing the sample input. It is therefore preferable to keep the cover in front of the sample input whenever sample measurement is not performed.

It is also possible that the device has means for detecting the position of the cover 21. The device may then remind the user to put the cover in a correct position. The detecting means may be based on detection of a small magnet placed in the movable cover. The detection means may also be based on optical detection. For example, the cover may have reflecting inner surface, and the reflection of the light from the light emitter 32 is detected. The position of the cover can thus be detected.

Additionally, the measurement device 10 includes preferably one or several wireless data transfer units 19. If radio data transmission is used the data transfer unit includes an antenna 39. Then the data transfer unit may be e.g. GSM 3G or 4G module of a cellular data transfer system to which a SIM card of a user may be connected. Such a unit may include a specific processor for controlling the data transfer. The communication capability in a mobile cellular system can be used, for example, for transferring measurement data and other data from a measurement device to a mobile phone. If a user is a child, the parents may receive the measurement data to their mobile phones. Also, if a user is an elderly person the relatives of the person may receive the measurement data.

The measurement device may also have a communication unit for Bluetooth communication, for example. Such wireless communication can be used with other measurement devices, such as a device measuring movement of the user or a device measuring heart pulse in order to receive other measurement data. This other measurement data can then be used as a further input in user's care program. However, it may be possible to connect such devices to the wired electrical connection 11 as well.

The data transfer means 11 and 19 may also transfer data between the measurement device and care devices. For example, a measurement device can transmit measurement data to an insulin injector and/or receive from an insulin injector information that the user has received an insulin dose. The measurement device may also use this information as an acknowledgement for the given activity instruction and as information which affects the reminders and activity instructions according to the care program of the user.

As described above, the measurement device may give reminders for the user. The time table for providing the reminders is stored in the memory 13 of the measurement device. The reminders may relate to performing a blood sample measurement, taking a dose of insulin, having a meal, having physical exercise, and resting.

Based on the measurement results, the measurement device may give an instruction for eating carbon hydrates or taking a dose of insulin. The processor 12 performs the appropriate reminders according to the stored time table. The user acknowledges the reminders and instructions with the corresponding input at the touch screen of the device, for example.

The measurement device may also store information on carbon hydrate contents and/or calorie contents of various foodstuff in the memory 13. The user may then check the carbon hydrate contents and/or calorie contents of the user's meal by selecting the type and amount of foodstuff at the selection menu of the device, whereby the processor 12 of the device calculates the amounts of carbon hydrate and/or calories of the defined food. The device may also store the results of the calculations, and the information can then be used as a diet history data. It is preferable that the same processor 12 is used for controlling measurements and calculating food contents, whereby several functions can be achieved with a single device including a small number of processor and memory components.

The device of FIG. 1 also includes an energy source 33, such as a rechargeable or disposable battery. A rechargeable battery may be charged via the USB connection, for example.

FIGS. 2a and 2b illustrate bottom view and a perspective view of an exemplary measurement device according to the invention. The sample input 24 and the USB connector 11 are located at the bottom side of the device. There is a sliding cover 21, which can be moved to cover either the sample input 24 or the electrical connection 11. The cover 21 has a protrusion 211 for manually moving the cover.

The measurement device of FIGS. 2a and 2b also has a touch screen 251 as a user interface. In a measurement device there is preferably stored parameters that belong to the user's care program, on basis of which parameters the device can remind the user of e.g. eating, physical exercise, rest, medication and/or measurement of blood glucose concentration at predetermined points of time. The measurement device may present this kind of reminders at the touch screen, and the reminders can be acknowledged by touching the touch screen. It is also possible to use tones or recorded/synthesized speech or vibration for reminding and instructing the user.

The measurement device may also have a protective casing made of silicon, for example. Such a casing may have means for attaching to a neckband, whereby the measurement device can be held hanging on the neck of a user.

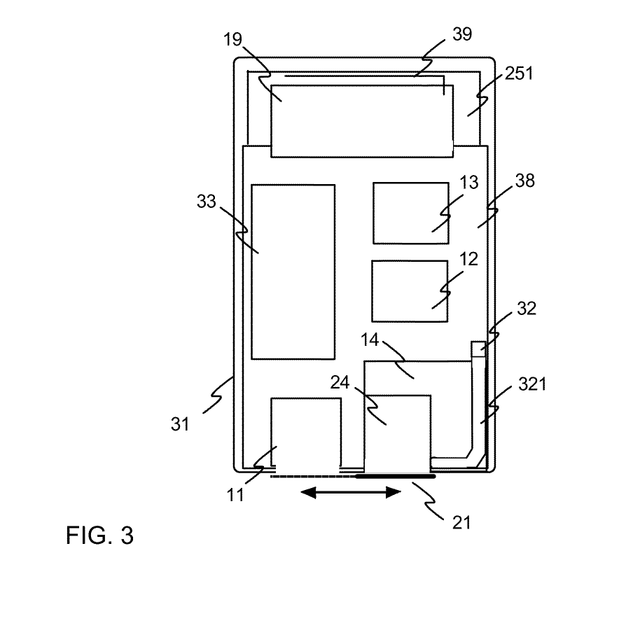

FIG. 3 illustrates a back view of the internal assembly of an exemplary measurement device according to the invention. The measurement device has an electronics module 38, which has the touch screen 251 at its front side, and other units/components at the back side. The measurement unit 14 is located at the bottom of the device, whereby the input 24 for a sample strip is located at the bottom side of the device. The electrical USB connector 11 is also located at the bottom side of the device, besides the sample input. The device has a sliding cover 21, which can be moved to cover either the electrical connector or the sample input.

The device has also a light emitter, such as an LED 32. The light from the LED is conveyed to the sample input aperture with a light guide 321. The light guide makes it possible to provide an optimal light distribution at the aperture 24.

The FIG. 3 also illustrates the processor 12, memory 13 and a battery 33. The wireless communication unit 19 and its antenna is located at the top area of the device. It is preferable to locate the measurement unit and the communication unit at opposite sides of the device in order to avoid interference from the transmitter to the measurement unit. It is, however, also possible to avoid interference by switching off the transmitter during sample measurement.

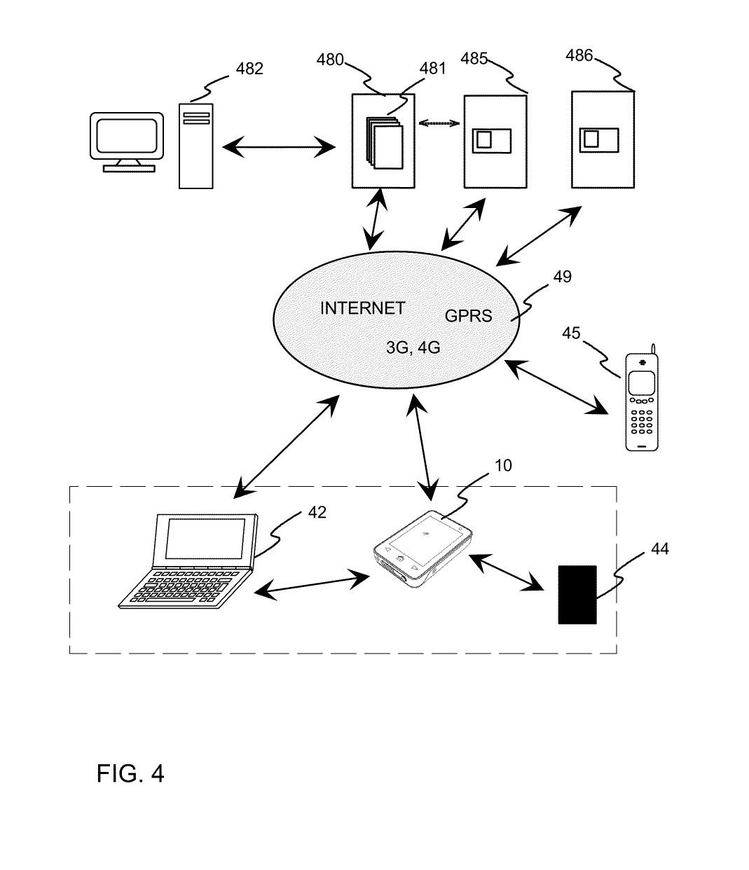

FIG. 4 illustrates a system where an exemplary measurement device 10 according to the invention is in connection with several types of equipment. The measurement device 10 can be connected to a user's laptop computer 42 with a wired, USB connection, in order to transfer measurement data and other user data, as well as update programs and parameters of the measurement device. The measurement device may also be in connection with other devices 44 of the user with a wired USB connection or wireless Bluetooth or NFC connection, for example. Such other devices may include other measurement devices, an insulin injector, etc. The measurement device preferably has a wireless connection with a cellular data transfer network in order to transfer data with mobile phones 45.

The measurement device may also be in connection with a public health care system. Data is transferred with a central unit 480 of a user information centre, using direct wireless communication through cellular data network, or using a computer 42 which is connected to the Internet. The above mentioned data may then be transferred between the measurement unit and the central unit. The central unit 480 comprises a database 481, into which personal information and information relating to the disease of the measurement device users is stored. The central unit can be e.g. a central computer of regional health care. The health care system includes terminal equipment 482 of the nursing staff, which terminal equipment is connected to the central computer 480. There may also be servers 485, 486 connected to the central computer, which servers may provide supplementary services for the users, such as games or other social communications. The purpose of the chat server is to assist communication of such people who have the same disease, such as diabetes. This way these people can exchange their experiences and support each other.

The central unit 480 can also be separate from other database of the health care, and which comprises only self care information relating to a certain disease, such as information on blood glucose concentration measurement and care monitoring. Nursing staff, such as a nurse and a doctor, has access to the information of the central unit. The access may take place e.g. with terminal equipment 482 after logging in the system.

Above some exemplary devices according to the invention have been described. The functionality according to the invention is achieved, in addition to the devices mentioned herein, by storing the programs which relate to the inventive functions and which control the processor/processors, into the memories of the devices. Programming a measurement device is known as such for a person skilled in the art, and such a person can implement the functions of the present invention on the basis of the description given here.

It must be noted that above only some embodiments of the solution according to the invention have been described. The principle of the invention can naturally be modified within the scope of protection determined by the patent claims, e.g. in details of implementation and areas of use.

The present invention is also not restricted to care of diabetes and monitoring of blood glucose concentration, but the invention can also be used for monitoring other diseases or for monitoring health. As an example, monitoring blood haemoglobin of athletes or people suffering from anaemia can be mentioned.

The present invention is neither restricted to use of, data transfer types, connector types or functions which were described in the above embodiments, but a person skilled in the art can design several alternative implementations in the frame of the inventive features. It is also possible to use various types of blocking means/covers as alternatives to the embodiment described above. For example, instead of sliding a cover may turn or rotate.

1-15. (canceled)

16. A user portable measurement device, which comprises a measurement unit for measuring a blood sample for e.g. blood glucose concentration, wherein the measurement unit has an input for providing the blood sample in the measurement unit, and the device has a user interface for presenting to a user information which is based on a measurement result, the user interface comprising at least a display and/or means for forming sound and/or vibration, characterised in that the device has means for blocking the sample input during time when the sample input is not under use.

17. A measurement device according claim 16, characterised in that the blocking means covers the sample input aperture of the device when the blocking means is in the position for blocking the sample input.

18. A measurement device according to claim 16, characterised in that the device has means for data transfer between the device and a computer.

19. A measurement device according to claim 18, characterised in that the means for the data transfer include an electrical connector, such as a micro-USB connector, for a wired connection between the device and a computer.

20. A measurement device according to claim 18, characterised in that the means for the data transfer include an electrical connector, such as a micro-USB connector, for a wired connection between the device and a computer, wherein blocking means are arranged to block the electrical connector when the sample input is not blocked.

21. A measurement device according claim 18, characterised in that the means for the data transfer include an electrical connector, such as a micro-USB connector, for a wired connection between the device and a computer, wherein blocking means are arranged to block the electrical connector when the sample input is not blocked, and the blocking means is a sliding cover, which in its first position covers the sample input, and in the second position covers the electrical connector.

22. A measurement device according to claim 16, characterised in that the device comprises storage means for storing information relating to measurement time table, and the device is adapted to give a reminder for the user at a moment when a measurement is to be performed.

23. A measurement device according to claim 16, characterised in that the measurement device has means for lighting the sample input when the sample input is in use.

24. A measurement device according to claim 23, characterised in that the device is adapted to switch ON the sample input light when the device gives a reminder for measurement.

25. A measurement device according claim 16, characterised in that the device has a processor which is adapted to control measurement and reminder functions and optionally data transfer functions.

26. A measurement device according to claim 16, characterised in that the device has a storage for storing information on carbon hydrate contents and/or calorie contents of various foodstuff, and a processor for calculating carbon hydrate contents and/or calorie contents of user defined food.

27. A measurement device according to claim 26, characterised in that the processor for controlling measurement and the processor for calculating food contents are substantially same processor.

28. A measurement device according to claim 16, characterised in that the device has a communication unit for wireless communication with a wireless communication network such as a mobile cellular communication network.

29. A measurement device according to claim 28, characterised in that the measurement device is adapted to transfer measurement data, user input data and/or reminder data to a wireless terminal via the wireless network.

30. A measurement device according to claim 28, characterised in that the communication unit has an antenna for wireless communication, the antenna and the measurement unit being positioned at opposite ends of the device housing in order to avoid mutual signal interference.
